(19) United States
(12) Patent Application Publication
(10) Pub. No.: US 2024/0241101 A1
(43) Pub. Date: Jul. 18, 2024

(54) SYSTEM AND METHOD FOR DETECTING DELETERIOUS MICROBIAL ACTIVITY IN HYDROGEN RESERVOIRS

(71) Applicant: Battelle Memorial Institute, Columbus, OH (US)

(72) Inventors: Morgan V. EVANS, Columbus, OH (US); Priya RAVI GANESH, San Jose, CA (US); Fadime KARA MURDOCH, Upper Arlington, OH (US); Dave ALBERTSON, Dublin, OH (US)

(21) Appl. No.: 18/413,325

(22) Filed: Jan. 16, 2024

Related U.S. Application Data
(60) Provisional application No. 63/480,238, filed on Jan. 17, 2023.

(51) Int. Cl. G01N 33/22; G01N 21/25

(52) U.S. Cl. CPC G01N 33/225; G01N 21/251; G01N 2333/195

(57) ABSTRACT

In an approach to detecting deleterious microbial activity in hydrogen reservoirs, a system includes a sample storage device; a biomarker indicator addition device; and a testing device. The system is further configured to receive a sample; add one or more biomarker indicators to the sample using the biomarker indicator addition device; and analyze the sample using the testing device to determine whether the sample contains one or more deleterious microorganisms.

### CROSS-REFERENCE TO RELATED APPLICATIONS

The present application claims the benefit of the filing date of U.S. Provisional Application Ser. No. 63/480,238, filed Jan. 17, 2023, the entire teachings of which application is hereby incorporated herein by reference.

### TECHNICAL FIELD

The present application relates generally to testing and monitoring systems and, more particularly, to a system, method, and apparatus for detecting deleterious microbial activity in hydrogen reservoirs.

### BACKGROUND

Energy production from renewable sources is subject to the availability of energy sources, such as wind or solar radiation. Weather and other factors can affect energy production from wind or solar, causing natural fluctuations in availability that can lead to shortages in energy production. One possibility to mitigate these shortages is to employ an energy storage scheme, such as geological storage. Hydrogen is a clean fuel that, when consumed in a fuel cell, produces only water. Hydrogen is an energy carrier that can be used to store, move, and deliver energy produced from other sources, but the inability to store large quantities of hydrogen is currently the main barrier to implementing an industrial-scale hydrogen economy.

Geological, or subsurface, storage facilities store inventory of liquid or gaseous fuels underground under pressure. The most common storage facilities are depleted reservoirs located at natural gas and oil fields, saline aquifers, and subterranean salt caverns. Geological storage is a possible option to store large quantities of hydrogen and therefore large amounts of energy over long timescales. Hydrogen can be stored using, for example, salt caverns or porous formations such as saline aquifers or depleted hydrocarbon fields.

### BRIEF DESCRIPTION OF THE DRAWINGS

Reference should be made to the following detailed description which should be read in conjunction with the following figures, wherein like numerals represent like parts.

FIG. 1 is an illustrative example of the problem statement consistent with the present disclosure.

FIG. 2 is an example of the method for detecting deleterious microbial activity in hydrogen reservoirs, consistent with the present disclosure.

FIG. 3 is an illustrative example of a system for detecting deleterious microbial activity in hydrogen reservoirs consistent with the present disclosure.

FIG. 4 is an expanded view of an example embodiment of a sandwich Lateral Flow Assay (LFA) consistent with the present disclosure.

FIGS. 5A-5D are a schematic representation of the working parts of the LFA of FIG. 4 consistent with the present disclosure.

### DETAILED DESCRIPTION

The present disclosure is not limited in its application to the details of construction and the arrangement of components set forth in the following description or illustrated in the drawings. The examples described herein may be capable of other embodiments and of being practiced or being carried out in various ways. Also, it may be appreciated that the phraseology and terminology used herein is for the purpose of description and should not be regarded as limiting as such may be understood by one of skill in the art. Throughout the present description, like reference characters may indicate like structure throughout the several views, and such structure need not be separately discussed. Furthermore, any particular feature(s) of a particular exemplary embodiment may be equally applied to any other exemplary embodiment(s) of this specification as suitable. In other words, features between the various exemplary embodiments described herein are interchangeable, and not exclusive.

Currently hydrogen storage is in early stages of exploration but will likely be increasing over the next several decades. With the increased production of hydrogen, large-scale seasonal storage systems will be needed, and abandoned hydrocarbon reservoirs, saline aquifers, and salt caverns are being investigated for hydrogen storage. Hydrogen storage in the subsurface is in early stages of exploration as a key component of hydrogen hubs in the envisioned large-scale deployment of a hydrogen economy in the United States.

A key unknown of hydrogen storage in porous media is the impact of biogeochemical interactions on hydrogen storage integrity by various hydrogen-consuming organisms present in potential reservoirs. Deleterious microorganisms exist in the subsurface and many of them are hydrogenotrophic and consume hydrogen, leading to storage depletion. These metabolisms may also sour hydrogen gas with methane and/or hydrogen sulfide, corrode the well with iron and sulfate reduction, or plug formations with biofilms, leading to decreased storage capacity. Disclosed herein is a system, method, and apparatus to indicate whether hydrogenotrophic bacteria are present in reservoirs for operators to determine likelihood of hydrogenotrophic microorganisms prior to or during storage operations. The approach involves potentially identifying a unique shared gene/protein between hydrogenotrophic microorganisms and developing a tool to rapidly detect/quantify the identified target biomarker(s) to indicate microorganisms that consume hydrogen on-site. The system, method, and apparatus disclosed herein are rapidly deployable and user friendly.

Storage of hydrogen may stimulate hydrogenotrophic organisms to metabolize and deplete hydrogen, produce unwanted metabolites that can sour the hydrogen by producing methane or hydrogen sulfide, can corrode the well via sulfate or iron reduction, or plug the formation through biofilm generation. Disclosed is a detection mechanism that may be an in-situ device or an on-site sampling system for ex situ analysis, for hydrogenotrophic bacteria present in reservoirs indicated for hydrogen storage. The disclosed system, method, and apparatus allow operators of wells to determine if deleterious microorganisms are present and capable of negatively impacting hydrogen storage and provide information to operators on courses of action to take if deleterious organisms are present. In various embodiments, the disclosed system, method, and apparatus may include, but are not limited to, rapid insights into the subsurface for storage operators in proposed regional integrated hydrogen hubs, which may be critical to site selection for large-scale hydrogen storage and may be critical to proactive remediation measures for preservation of the storage integrity of the subsurface system and minimize unacceptable losses. Potential adjacent markets may include microbial-induced corrosion management in hydrogen transportation and complement subsurface operational integrity management for carbon capture, utilization, and storage (CCUS) and geothermal extraction technologies.

Characterizing and monitoring these interactions is critical for operational management to minimize unacceptable losses in the subsurface system. Disclosed herein is a system, method, and apparatus for detecting deleterious microbial activity in hydrogen reservoirs prior to/during storage operations. The disclosure targets hydrogen consuming bacteria that will specifically harm hydrogen reservoirs, as opposed to similar inventions which only target metabolisms known to traditional oil and gas reservoirs (e.g., sulfate reduction). However, the application of these types of devices to hydrogenotrophic bacteria is a new idea.

Disclosed herein is a rapid microbe characterization and monitoring system, method, and apparatus for rapid determination of expected microbial activity in the subsurface and potential associated losses to the hydrogen. This is accomplished by using a unique shared gene/protein between target organisms and a rapid tool to detect and/or quantify the target biomarker.

Biomarker targets explored through bioinformatics were identified as highly conserved and expressed at high levels within a large group of hydrogen metabolizing microorganisms. Seven protein candidates were found to be conserved at up to 71% of genomes within a test group of hydrogen metabolizing microbes, such as Methanomicrobia.

In some embodiments, the disclosed system may be a quantitative method, i.e., a spectrophotometric method. Spectrophotometry is a tool for the quantitative analysis of molecules depending on how much light is absorbed by colored compounds. Spectrophotometry is a quantitative measurement technique to investigate the optical properties of materials over a wide wavelength range, from the ultraviolet to the visible and infrared spectral regions. It involves measuring the ratio of two ratiometric quantities as a function of wavelength. Biomarkers unique to target proteins/enzymes of hydrogenotrophic microorganisms can be incorporated into a test vial. This method provides quantitative characterization of the types and proportions of the hydrogenotrophic microorganisms that can inform the site operator/owner of the risk profile relevant to storing hydrogen at the particular site.

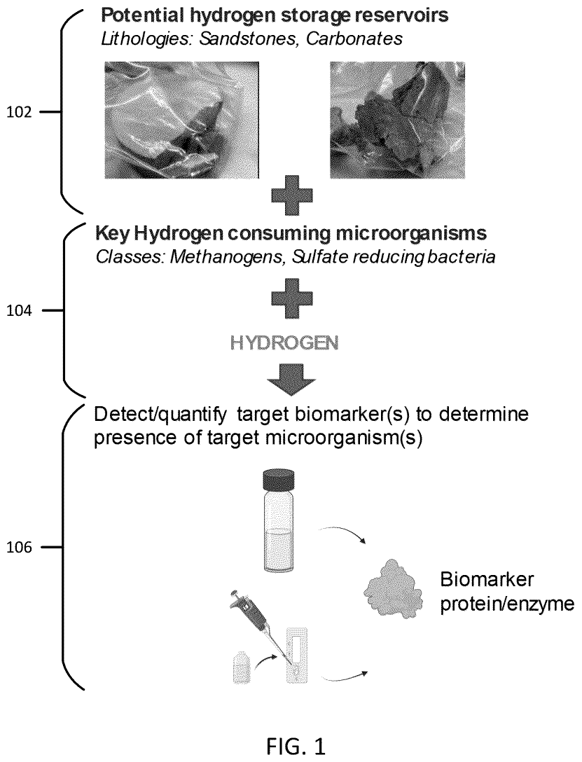

FIG. 1 is an illustrative example of the problem statement consistent with the present disclosure. Potential geological storage sites 102 may include porous substances such as sandstone and carbonates. The issue 104 is that operations introducing the hydrogen in storage facilities that may interact with in-situ hydrogen consuming microorganisms, such as methanogens and/or sulfate reducing bacteria which may result in hydrogen consumption. In addition, as described above, these deleterious microorganisms may sour hydrogen gas with methane and/or hydrogen sulfide, corrode the well with iron and sulfate reduction, or plug formations with biofilms. All of these may lead to decreased hydrogen storage capacity. Solution 106 illustrates the detection and/or quantification of one or more target biomarkers to determine the presence of target organisms and the resultant potential losses. One embodiment of the details of solution 106 are illustrated in the example of method 200 below.

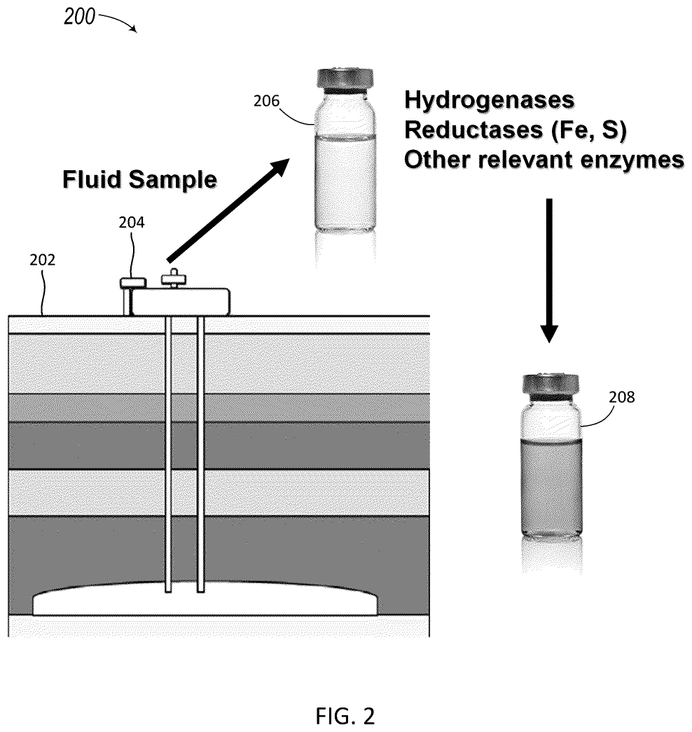

FIG. 2 is an example of method 200 for detecting deleterious microbial activity in hydrogen reservoirs, consistent with the present disclosure. In the example of FIG. 2 a sample 206, which may include a fluid sample and/or rock cuttings, is drawn from the storage reservoir 202 via a sample collection device 204. In various embodiments, the sample collection device 204 may be a storage unit, e.g., a bottle with a sealed lid, where a sample will be added to the bottle, then a sachet will be placed into the bottle with samples to capture the oxygen in the bottle to create an anaerobic environment and then the bottle will be sealed with a lid to prevent oxygen intrusion.

Testing applications include preinjection reservoir characterization as part of screening evaluation of potential sites as well as during hydrogen storage operations as part of performance assurance monitoring. Fluid samples may be collected for microbial testing and can be part of routine fluid sampling/analysis. Fluid sampling procedures should follow recommended practices typical for sampling oil and gas wells to obtain “representative” samples of the fluid or fluids found in the reservoir at initial conditions. In an embodiment, the operation may involve establishing zonal isolation and cleanup as needed of the injection interval(s) that need to be evaluated. Once acceptable contamination/sand-free stable flow is established, high quality fluid samples are collected. The samples for microbial testing should be collected in sterile, precleaned sample chambers/bottles that are attached to the sampler probe of the sample collection device 204. The bottles should just be filled to the brim (as little headspace as possible) and sealed. The samples should be handled with care to ensure the samples are unaltered from reservoir conditions. This may include wearing gloves to ensure that sterile conditions are maintained and to avoid any contamination.

Biomarker indicator specific to the biomarker protein/enzyme is added to sample 206 that indicate the presence of the deleterious microorganisms. The result 208 is the sample 206 that indicates the presence of the deleterious microorganisms, in this example, by the change in color of the liquid.

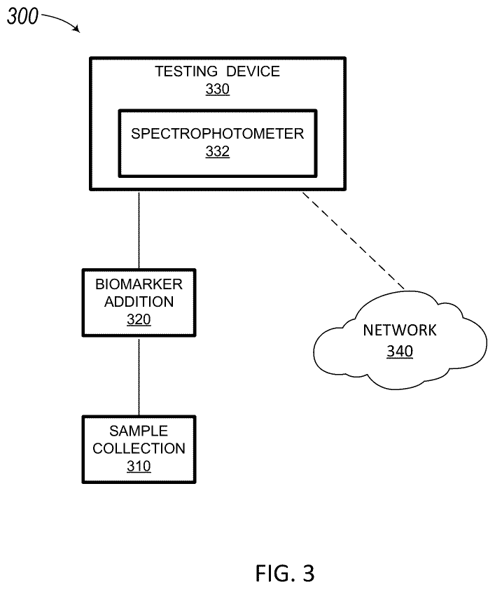

FIG. 3 is an illustrative example of a system 300 for detecting deleterious microbial activity in hydrogen reservoirs consistent with the present disclosure. FIG. 3 represents only one illustrative example system for detecting deleterious microbial activity in hydrogen reservoirs. Many other embodiments of the present disclosure are possible, as would be known to a person of skill in the art.

In the illustrative example of FIG. 3, system 300 includes sample collection 310. In some embodiments, sample collection 310 may be incorporated into the storage facility such as sample collection device 204 of FIG. 2. Once the sample has been collected, the sample is prepared by adding specific biomarkers targeting specific protein/enzyme unique to hydrogenotrophic microorganisms to the sample by biomarker addition 320. In some embodiments, biomarker addition 320 may be an automated biomarker addition device. In some other embodiments, biomarker addition 320 may be a manual process. In yet some other embodiments, biomarker addition may be by any suitable device or method as would be known to a person of skill in the art.

In the illustrative example of FIG. 3, system 300 includes testing device 330, which further includes spectrophotometer 332. As discussed above, spectrophotometry is a tool for the quantitative analysis of molecules depending on how much light is absorbed by colored compounds. A spectrophotometer is a device that precisely measures electromagnetic energy at specific wavelengths of lights, which uses these measurements to identify colors and determine how much of each color is present in a ray of light.

Once the biomarker has been added to the sample, the prepared sample is analyzed by testing device 330. In some embodiments, testing device 330 may be portable or located at, or integral with, the storage facility.

In the illustrative example of FIG. 3, testing device 330 incorporates spectrophotometer 332 to analyze the color of the light from the sample once the biomarker indicator has been added to determine the presence of deleterious microorganisms in the sample. As explained in FIG. 2 above, the biomarker indicators specific to the biomarkers such as enzymes that are added to the sample indicate the presence of the deleterious microorganisms. Spectrophotometer 332 analyzes the sample to determine whether the deleterious microorganisms are present in the sample and, if so, the abundance of these microorganisms based on a color intensity of the final color development. The results of the analysis may be provided to a user by testing device 330. The results may also include a risk profile relevant to storing hydrogen at the particular site and can supply potential remediation measures for proceeding with intended storage operations.

In some embodiments, a single biomarker indicator may be added to the sample to determine whether any of the deleterious microorganisms are present. In some other embodiments, multiple biomarker indicators may be added to the sample to detect multiple deleterious microorganisms. In yet some other embodiments, the sample may be divided into multiple samples, where each sample is then combined with a specific biomarker indicator to allow detection of multiple deleterious microorganisms.

Methodologically, environmental sampling typically requires a lysis buffer component to release intracellular targets, which could be included as part of the assay. Additionally, these assays must be performed with a dilution series to achieve accurate results and reduce false-negatives due to over-saturation, which are common when assaying unknown concentrations of sample. Thus, in an embodiment, the disclosed system may be a kit-based system that includes the material components, test vial(s) and biomarker indicator(s) to run multiple assays in tandem.

In some embodiments, testing device 330 may include network 340. Network 340 can be, for example, a telecommunications network, a local area network (LAN), a wide area network (WAN), such as the Internet, or a combination of the three, and can include wired, wireless, or fiber optic connections. Network 340 can include one or more wired and/or wireless networks that are capable of receiving and transmitting data, voice, and/or video signals, including multimedia signals that include voice, data, and video information. In general, network 340 can be any combination of connections and protocols that will support communications between testing device 330 and other devices (not shown) within distributed system 300. In some embodiments, network 340 may be used to interact with a remote user, or to provide results to a user from a remote testing facility.

In an embodiment, the disclosed system may be a qualitative method, i.e., absence/presence. Antibodies developed against these unique targets can be incorporated into a variety of detection formats for the development of assay kits, or other more elaborate detection media. Simple, direct measurements during long term monitoring efforts can increase the likelihood of discovery of a contamination event. With these unique antibodies in hand commonplace detection methods, such as lateral flow assays (LFA) or Enzyme-Linked Immunosorbent Assays (ELISA), can be utilized to efficiently and rapidly probe for these unique targets in a live testing environment.

The “sandwich” lateral flow assay is a common format of LFA that utilizes three antibodies: a primary detection antibody, a capture antibody, and a species-targeted control antibody. Typically, the primary and capture antibodies are raised in different species to minimize species cross-reactivities. The primary detection antibody is often, but not exclusively, a mouse monoclonal antibody and is conjugated to a suitable detection probe. In the situation of medical diagnostics testing (e.g., pregnancy test tickets or tests for COVID-19) colloidal gold is often used due to its non-reactivity, stable shelf life and vibrant color. Other probes, such as fluorescent and/or luminescent up-converting nanoparticles, could also be used offering an increase in detectability in combination with other optical systems. However, these materials could be included at the cost of more complicated optical equipment for measurement, whereas colloidal gold can be detected by eye.

The species control antibody is often a polyclonal antibody detecting the species-identifying markers of the primary detection antibody's host animal. This serves as an important internal control to observe poor test outcomes, such as non-specific binding and over-saturation of the test strip. The Hook effect is a well-known outcome of over-saturation and is solved by running a dilution series of samples on several tickets.

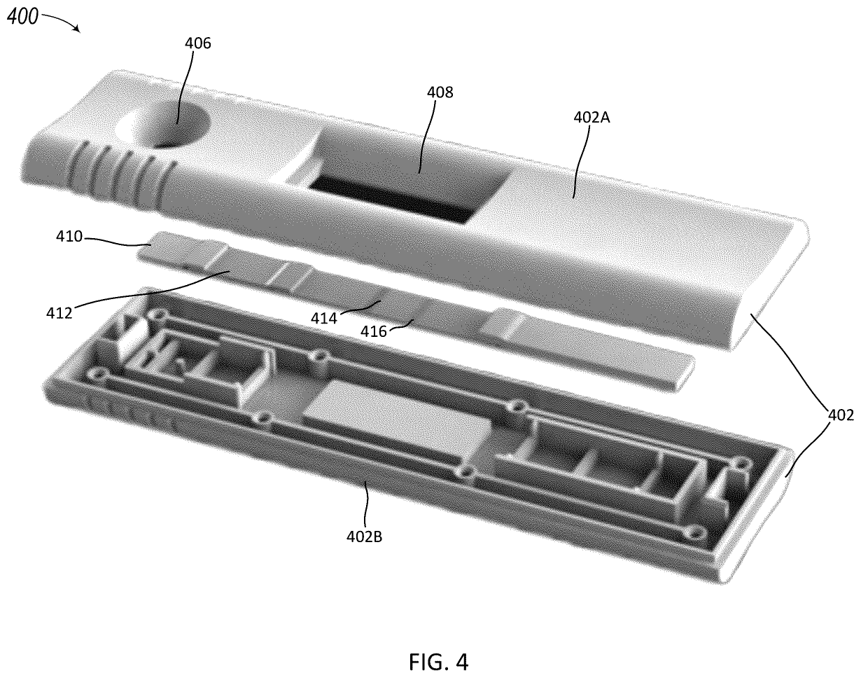

FIG. 4 is an expanded view of an example embodiment of a sandwich LFA 400 consistent with the present disclosure. LFA 400 is an apparatus that includes a case 402 consisting of a top section 402A coupled to a bottom section 402B, with test strip 410 disposed between the top section 402A and the bottom section 402B. Top section 402A includes sample port 406, which may be a circular cavity with an opening configured to allow a sample, e.g., a liquid, to enter the LFA 400 and to be deposited on the test strip 410. The top section 402A also includes a result window 408, which allows a user to view the results of the test. Test strip 410 includes a primary detector 412, a test line 414 containing the capture antibodies, and a control line 416 containing anti-species antibodies.

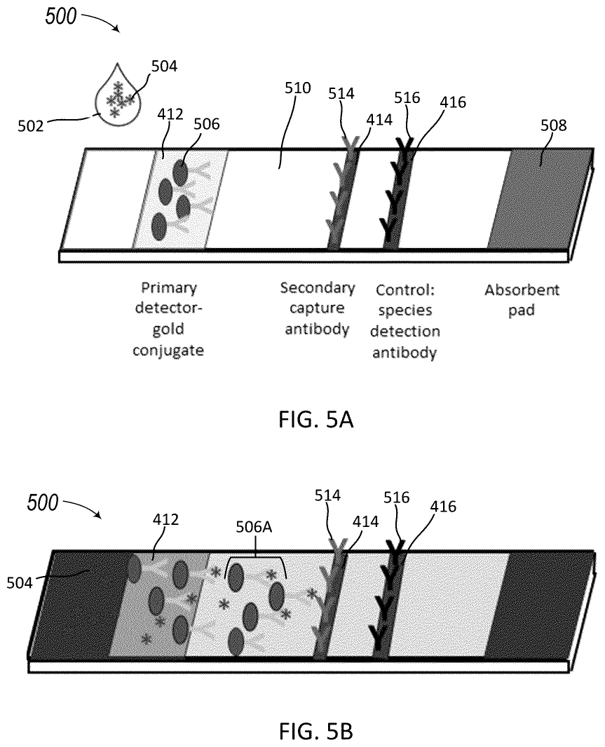

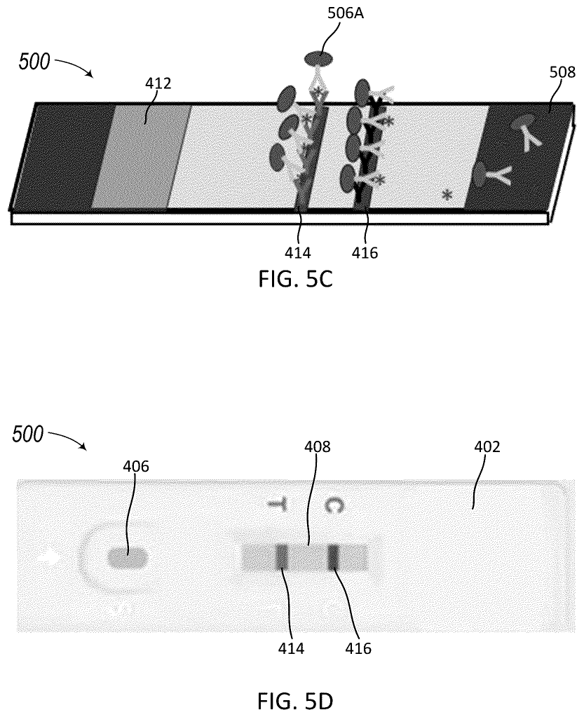

FIGS. 5A-5D are a schematic representation of the working parts of test strip 500 of a sandwich LFA, such as the test strip 410 of the LFA 400 of FIG. 4, consistent with the present disclosure. Assays such as these can be manufactured at scale and provide a rapid detection of the defined biomarker targets from methanogenic microbial species of concern.

FIG. 5A illustrates the test strip 500 as a sample 502 is applied to the LFA. The sample 502 contains target antigens 504. Test strip 500 comprises substrate 510, which may be, for example, a nitrocellulose membrane, and the primary detector 412 which may be, for example, a gold conjugate, and which contains the primary antibodies 506. The test strip 500 also includes test line 414 which contains secondary capture antibodies 514 and the control line 416 which contains species detection antibodies 516. The test strip 500 also includes an absorbent pad 508.

In FIG. 5B, the test strip 500 has been hydrated and the gold-labeled primary antibodies 506 bind the target antigens 504 in the sample, forming primary gold target-antigen complexes 506A. Liquid flow wicks the bound target-antigen complexes 506A to the test line 414 and the control line 416.

In FIG. 5C, the secondary capture antibodies 514 and the species detection antibodies 516 bind the target-antigen primary gold complexes 506A, with the remaining unbound complexes, i.e., the target antigens 504 and the primary antibodies 506, collecting at the absorbent pad 508.

FIG. 5D is an example of the expected results from a positive test. It should be noted that both the test line 414 and the control line 416 are clearly visible in result window 408.

Individual LFA tickets can only detect a single biomarker target, however these kits could be expanded to include a panel of targets, where each LFA in the panel contains a different target than each other LFA. Seven targets have been defined that could serve as detection biomarkers across a large group of hydrogen metabolizing bacteria. A panel of targets could expand the identification range of the assays to include many tangentially related species that could likewise damage hydrogen reservoirs.

In an embodiment, the LFA may be used in conjunction with spectrophotometric assays. Using the LFA in conjunction with spectrophotometric assays offers an improved method for detection and increases confidence in the results. Spectrophotometric analysis of reaction results (colorimetric measurements from enzymatic output, or a decrease in hydrogen content) could be caused by other coincidental factors, however in conjunction with positive LFA results this offers a stronger case for presumptive detection of the potential for microbial-induced losses of injected hydrogen in the reservoir or subsurface formation being tested. Positive LFA results can thus be supplemented by spectrophotometric analysis to form a comprehensive test result for a timely and complete onsite microbe characterization and monitoring system targeting hydrogen storage applications in porous media in the subsurface. In this embodiment, once the sample is acquired from the hydrogen reservoir, the sample may be divided into a first portion to be used with the LFA, and the remaining portion is used with the spectrophotometric assay.

In some embodiments, the method and system disclosed herein may be used for site screening. These embodiments may entail testing for microbial activity at different depths corresponding to potential reservoirs or formations at a site and providing a ranking to enable the operator to select the best option for the target site.

According to one aspect of the disclosure, there is provided a system. The system includes a sample storage device; a biomarker indicator addition device; and a testing device. The system is further configured to: receive a sample; add one or more biomarker indicators to the sample using the biomarker indicator addition device; and analyze the sample using the testing device to determine whether the sample contains one or more deleterious microorganisms.

According to another aspect of the disclosure, there is provided an apparatus for detecting deleterious microbial activity in hydrogen reservoirs, the apparatus including: a case, the case further including: a bottom section; a top section; and a sample port and a result window disposed in the top section; a test strip disposed within the case, the test strip further including: a primary detector; a test line; and a control line, where: the test line and the control line configured to bind a target antigen; and the test line and the control line disposed to be visible through the sample port.

According to yet another aspect of the disclosure, there is provided a method, the method including: a sample storage device; a biomarker indicator addition device; a testing device; and a Lateral Flow Assay (LFA); the system further configured to: collect a sample from a storage reservoir; apply a first portion of the sample to the LFA to test the sample; receive a first result from the LFA; add one or more biomarker indicators to a remaining portion of the sample using the biomarker indicator addition device; analyze the remaining portion of the sample using the testing device to determine a second result of whether the sample contains one or more deleterious microorganisms; and combine the first result from the LFA and the second result from the testing device to form a comprehensive test result.

As used in this application and in the claims, a list of items joined by the term “and/or” can mean any combination of the listed items. For example, the phrase “A, B and/or C” can mean A; B; C; A and B; A and C; B and C; or A, B and C. As used in this application and in the claims, a list of items joined by the term “at least one of” can mean any combination of the listed terms. For example, the phrases “at least one of A, B or C” can mean A; B; C; A and B; A and C; B and C; or A, B and C.

The foregoing description of example embodiments has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto.

Embodiments of the methods described herein may be implemented using a controller, processor and/or other programmable device. To that end, the methods described herein may be implemented on a tangible, non-transitory computer readable medium having instructions stored thereon that when executed by one or more processors perform the methods. Thus, for example, the memory may store instructions (in, for example, firmware or software) to perform the operations described herein. The storage medium may include any type of tangible medium, for example, any type of disk optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, magnetic or optical cards, or any type of media suitable for storing electronic instructions.

It will be appreciated by those skilled in the art that any block diagrams herein represent conceptual views of illustrative circuitry embodying the principles of the disclosure. Similarly, it will be appreciated that any block diagrams, flow charts, flow diagrams, state transition diagrams, pseudocode, and the like represent various processes which may be substantially represented in computer readable medium and so executed by a computer or processor, whether or not such computer or processor is explicitly shown. Software modules, or simply modules which are implied to be software, may be represented herein as any combination of flowchart elements or other elements indicating performance of process steps and/or textual description. Such modules may be executed by hardware that is expressly or implicitly shown.

The functions of the various elements shown in the figures, including any functional blocks labeled as a controller or processor, may be provided through the use of dedicated hardware as well as hardware capable of executing software in association with appropriate software. The functions may be provided by a single dedicated processor, by a single shared processor, or by a plurality of individual processors, some of which may be shared. Moreover, explicit use of the term controller or processor should not be construed to refer exclusively to hardware capable of executing software, and may implicitly include, without limitation, digital signal processor (DSP) hardware, network processor, application specific integrated circuit (ASIC), field programmable gate array (FPGA), read-only memory (ROM) for storing software, random access memory (RAM), and non-volatile storage. Other hardware, conventional and/or custom, may also be included.

The term “coupled” as used herein refers to any connection, coupling, link, or the like by which signals carried by one system element are imparted to the “coupled” element. Such “coupled” devices, or signals and devices, are not necessarily directly connected to one another and may be separated by intermediate components or devices that may manipulate or modify such signals.

Unless otherwise stated, use of the word “substantially” may be construed to include a precise relationship, condition, arrangement, orientation, and/or other characteristic, and deviations thereof as understood by one of ordinary skill in the art, to the extent that such deviations do not materially affect the disclosed methods and systems. Throughout the entirety of the present disclosure, use of the articles “a” and/or “an” and/or “the” to modify a noun may be understood to be used for convenience and to include one, or more than one, of the modified noun, unless otherwise specifically stated. The terms “comprising,” “including,” and “having” are intended to be inclusive and mean that there may be additional elements other than the listed elements.

Although the methods and systems have been described relative to a specific embodiment thereof, they are not so limited. Obviously, many modifications and variations may become apparent in light of the above teachings. Many additional changes in the details, materials, and arrangement of parts, herein described and illustrated, may be made by those skilled in the art.

What is claimed is:

1. A system for detecting deleterious microbial activity in hydrogen reservoirs, the system comprising:
a sample storage device;
a biomarker indicator addition device; and
a testing device;
the system further configured to:
receive a sample;
add one or more biomarker indicators to the sample using the biomarker indicator addition device; and
analyze the sample using the testing device to determine whether the sample contains one or more deleterious microorganisms.

2. The system of claim 1, wherein the testing device further comprises a spectrophotometer.

3. The system of claim 2, wherein analyze the sample using the testing device to determine whether the sample contains the one or more deleterious microorganisms further comprises:
prepare the sample for spectrophotometry;
analyze the prepared sample using the spectrophotometer; and
determine whether the sample contains the one or more deleterious microorganisms based on a result from the spectrophotometer.

4. The system of claim 2, further comprising:
responsive to determining that the sample contains the one or more deleterious microorganisms, determine an amount of the one or more deleterious microorganisms in the sample.

5. A method for detecting deleterious microbial activity in hydrogen reservoirs, the method comprising:
collecting a sample from the hydrogen reservoir;
adding one or more biomarker indicators to the sample; and
analyzing the sample to determine whether the sample contains one or more deleterious microorganisms.

6. The method of claim 5 wherein the sample is collected using a sample collection device and stored in a sample storage device.

7. The method of claim 5 wherein the one or more biomarker indicators specific to different biomarkers are added to the sample using an automated biomarker addition device.

8. The method of claim 5 wherein the sample is analyzed using a testing device.

9. The method of claim 8 wherein the testing device further comprises a spectrophotometer.

10. The method of claim 9 wherein the sample is analyzed using the spectrophotometer to determine whether the sample contains the one or more deleterious microorganisms.

11. The method of claim 10 further comprising:
responsive to determining that the sample contains the one or more deleterious microorganisms, determine an amount of the one or more deleterious microorganisms in the sample.

12. An apparatus for detecting deleterious microbial activity in hydrogen reservoirs, the apparatus comprising:
a case, the case further comprising:
a bottom section;
a top section; and
a sample port and a result window disposed in the top section;
a test strip disposed within the case, the test strip further comprising:
a primary detector;
a test line; and
a control line, wherein:
the test line and the control line configured to bind a target antigen; and
the test line and the control line disposed to be visible through the sample port.

13. The apparatus of claim 12, further comprising:
primary antibodies disposed on the primary detector;
secondary capture antibodies disposed on the test line; and
species detection antibodies disposed on the control line.

14. The apparatus of claim 13, wherein:
target antigens from a sample are bound to the primary antibodies to form target-antigen complexes.

15. The apparatus of claim 14, wherein:
liquid flow of the sample wicks the target-antigen complexes to the test line and the control line; and
the target-antigen complexes are bound to the secondary capture antibodies on the test line and the species detection antibodies on the control line.

16. A system for detecting deleterious microbial activity in hydrogen reservoirs, the system comprising:
a sample storage device;
a biomarker indicator addition device;
a testing device; and
a Lateral Flow Assay (LFA);
the system further configured to:
collect a sample from a storage reservoir;
apply a first portion of the sample to the LFA to test the sample;
receive a first result from the LFA;
add one or more biomarker indicators to a remaining portion of the sample using the biomarker indicator addition device;
analyze the remaining portion of the sample using the testing device to determine a second result of whether the sample contains one or more deleterious microorganisms; and
combine the first result from the LFA and the second result from the testing device to form a comprehensive test result.

17. The system of claim 16, wherein the testing device further comprises a spectrophotometer.

18. The system of claim 17, wherein analyze the remaining portion of the sample using the testing device to determine the second result of whether the sample contains the one or more deleterious microorganisms further comprises:
prepare the remaining portion of the sample for spectrophotometry;
analyze the prepared remaining portion of the sample using the spectrophotometer; and
determine whether the remaining portion of the sample contains the one or more deleterious microorganisms based on a result from the spectrophotometer.

19. The system of claim 16, wherein the LFA further comprises:
a case, the case further comprising:
a bottom section;
a top section; and
a sample port and a result window disposed in the top section;
a test strip disposed within the case, the test strip further comprising:
a primary detector;
a test line; and
a control line, wherein:
the test line and the control line configured to bind a target antigen; and
the test line and the control line disposed to be visible through the sample port.

20. The system of claim 16, wherein the LFA further comprises:
a panel of targets, wherein the panel of the targets comprises a plurality of LFAs, and wherein each LFA of the plurality of LFAs contains a different target than each other LFA of the plurality of LFAs.
